(19) United States
(12) Patent Application Publication
(10) Pub. No.: US 2020/0347380 A1
(43) Pub. Date: Nov. 5, 2020

(54) Separation Device, and Method of Use, to Remove PCR Inhibitors from Whole Blood and Serum Samples

(71) Applicant: Entopsis, LLC, Miami, FL (US)

(72) Inventors: Francis Buan Hong Lim, Miramar, FL (US); Obdulio Piloto, Coral Gables, FL (US); Ian Shen-Yi Cheong, Finksburg, MD (US)

(73) Assignee: Entopsis, LLC, Miami, FL (US)

(21) Appl. No.: 16/770,580

(22) PCT Filed: Dec. 5, 2018
(86) PCT No.: PCT/US18/64143

§ 371 Date: Jun. 5, 2020

Related U.S. Application Data
(60) Provisional application No. 62/595,573, filed on Dec. 6, 2017.

(51) Int. Cl. C12N 15/10; B01L 3/00; C12Q 1/6806

(52) U.S. Cl. CPC C12N 15/1006; B01L 3/5021; B01L 2300/123; B01L 2300/0838; B01L 2300/0609; C12Q 1/6806

(57) ABSTRACT

A method and separation device for removing PCR inhibitors from whole blood or plasma/serum, is provided. The disclosed embodiments include the separation device; and system for removing PCR inhibitors and separating PCR inhibitors from nucleic acids, such as DNA or RNA, and the other components found in whole blood or plasma/serum samples, and methods of making and using the same, so that uncontaminated DNA/RNA is replicated. The separation device comprises: a receptacle for holding whole blood, serum or plasma samples; and a physical substrate for binding and removing one or more PCR inhibitors from the sample matrices, and glass capillary tubes. Physical substrates comprise, e.g. a crosslinked copolymer comprising acrylamide and N,N′-Methylenebisacrylamide, and one or more derivatives of acrylamide and acrylate with glycerol; and non-acrylamide based polymers such as sodium alginate or polyvinyl alcohol with agarose. And the receptacle comprises, e.g.: a 96-well plate, or a round-bottomed micro-centrifuge tube.

### CROSS-REFERENCE TO RELATED APPLICATION

The present application claims priority to U.S. provisional application Ser. No. 62/595,573 filed on Dec. 6, 2017, the entire contents of which are incorporated herein by reference.

### FIELD OF THE INVENTION

The present disclosure relates to a method and device for binding and thereby depleting PCR inhibitors found in whole blood or in plasma/serum.

### COPYRIGHT NOTICE

A portion of the disclosure of this provisional patent application document contains material that is subject to copyright protection. The copyright owner-inventor-assignee has no objection to the facsimile reproduction by anyone of the patent document or the patent disclosure, as it appears in the Patent and Trademark Office patent files or records, but otherwise reserves all copyright rights whatsoever. All copyrights disclosed herein are the property of their respective owners.

### TRADEMARKS DISCLAIMER

The product names used in this document are for identification purposes only. All trademarks and registered trademarks are the property of their respective owners.

### BACKGROUND OF THE INVENTION

The Polymerase Chain Reaction (PCR) is a revolutionary method developed in the 1980s that enables the amplification of a single copy, or a few copies of, a piece of DNA across several orders of magnitude, leading to the synthesis of thousands to millions of copies of a particular DNA sequence. PCR involves the exponential amplification of DNA templates by a DNA polymerase using specific primer molecules. The Reverse Transcription-Polymerase Chain Reaction (RT-PCR) is a variation of PCR in which RNA is first transcribed into cDNA by reverse transcriptase, and the resultant cDNA is subsequently PCR-amplified. PCR and its variants are powerful tools which can be used for the diagnosis of diseases in a large variety of sample types such as biological materials (organs, blood, body fluids etc.), environmental samples (water, soil, air etc.) and food matrices (milk, meat, seafood, fruits, vegetables etc.). In practice however, PCR is often inhibited and suppressed by one or more of a heterogeneous group of PCR inhibitory substances (e.g. see Table 1).

TABLE 1
Selected sample matrices or types and their identified PCR inhibitors (27).

| Sample Matrix | Contained inhibitors | References |
|---|---|---|
| Clinical specimens | Antiviral substances (e.g. acyclovir), | Al-Soud and Rådström (2001); Burkardt |
| (e.g. blood; muscle | Haemoglobin, Heparin, Hormones, | (2000); Rådström et al. (2004); |
| tissues) | IgG, Lactoferrin, Myoglobin | Yedidag et al. (1996) |
| Stool | Complex polysaccharides, Bile salts, | Kreader (1996); Monteiro et al. (1997); |
| | Lipids, Urate | Rådström et al. (2004); Chaturvedi et al. |
| | | (2008) |
| Seafood, bivalves, | Algae, Glycogen, Polysaccharides | Atmar et al. (1993, 1995); Richards (1999) |
| oysters | | |
| Berries | Phenols, Polysaccharides | Seeram et al. (2006); Wei et al. (2008) |
| Plants | Pectin, Polyphenols, Polysaccharides, | Demeke and Adams (1992); Henson and |
| | Xylan | French (1993); John (1992); Sipahioglu et al. |
| | | (2006); Su and Gibor (1988); Wan and |
| | | Wilkins (1994); Wei et al. (2008); Wilkins |
| | | and Smart 1996 |
| Cheese, milk | Proteases (e.g. plasmin), Calcium | Bickley et al. (1996); Powell et al. (1994); |
| | ions, | Rossen et al. (1992) |
| Water, | Debris, Fulmic acids, Humic acids, | Abbaszadegan et al. (1993); Ijzerman et al. |
| environment | Humic material Metal ions, | (1997) |
| | Polyphenol | |
| Palaeobiology, | Bone dust, Coprolite Peat extract, | Baar et al. (2011) |
| archaeology, | Clay-rich soil | |
| forensic | | |

These PCR inhibitors generally exert their effects through direct interference with thermostable DNA polymerases or interaction with DNA. Direct binding of PCR inhibitors to single-stranded and double-stranded DNA prevents DNA amplification; but, it can also facilitate co-purification of inhibitor and DNA. Inhibitors can also interact directly with DNA polymerases to block enzyme activity (e.g. changing or degrading the polymerase's protein structure or blockage of its catalytic site). Moreover, DNA polymerases have cofactor requirements that are also targets of inhibition.

Magnesium is a critical cofactor, and inhibitors that interfere with binding of Mg2+ to the polymerase or reduce Mg2+ availability, also serve to limit the application of PCR (e.g. see Table 2).

TABLE 2
Examples of PCR inhibitors and their inhibition mechanisms (28).

| Inhibitor | Mechanism of Action | References |
|---|---|---|
| Polyphenols, Polysaccharides | Co-precipitation with nucleic acid; | John (1992), Sipahioglu et al. |
| | reduction in the ability to | (2006), Su and Gibor (1988), Wan and |
| | resuspend precipitated RNA | Wilkins (1994) and Wilkins and |
| | | Smart (1996) |
| Bacterial cells Cell Debris | Degradation/sequestration of | Burkardt (2000), Katcher and |
| Detergents PCR additives | nucleic acids | Schwartz (1994), Peist et al. |
| Proteins | | (2001), Rossen et al. (1992), |
| Polysaccharides | | Weyant et al. (1990) and Wilson |
| Salts | | (1997) |
| Solvents | | |
| Polyphenols Polysaccharides | Cross-linking with nucleic acids; | John (1992), Opel et al. (2010) |
| Humic acids Collagen | change of chemical properties of | and Wilkins and Smart (1996) |
| Melanin | nucleic acids | |
| Humic acid | Binding/adsorption to nucleic acid | Abbaszadegan et al. (1993) |
| Humic matter | and enzymes | |
| Haematin | Incomplete melting of DNA | Opel et al. (2010) |
| Indigo | | |
| Metal Ions | Reduction in specificity of primers | Abbaszadegan et al. (1993) |
| Detergents | Degradation of polymerases | Powell et al. (1994), Rossen et al. |
| Proteases | | (1992), Saulnier and Andremont |
| Urea | | (1992) and Wilson (1997) |
| Calcium | Inhibition of DNA polymerase or | Al-Soud et al. (2000a), Al-Soud |
| Collagen | reverse transcriptase activity | and Rådström (1998), Eckhart et |
| Haematin | | al. (2000), Opel et al. (2010), |
| Herbal metabolites | | Peist et al. (2001) and Wilkins |
| IgG | | and Smart (1996) |
| Melanin | | |
| Myoglobin | | |
| Polysaccharides | | |
| Sodium | | |
| Tannic acid | | |
| Polyphenols | Chelation of metal ions | Abbaszadegan et al. (1993) and |
| Tannic acid | | Opel et al. (2010) |
| EDTA | Chelation of metal ions including | Rossen et al. (1992) |
| | Mg+ | |
| Calcium ions | Competition with co-factors of the | Bickley et al. (1996), Opel et al. |
| | polymerase | (2010) |
| Antiviral substances (e.g. | Competition with nucleotides | Yedidag et al. (1996) |
| acyclovir) | inhibition of DNA elongation | |
| Exogenic DNA | Competition with template | Tamariz et al. (2006) |

In clinical samples such as blood, urine or stool, the PCR inhibitors inherent in each sample type inhibit PCR with different mechanisms. For blood samples, substances like hemoglobin, lactoferrin, IgG antibodies, and anticoagulants such as heparin are presumed to directly affect RNA/DNA and not the enzymes in the reaction (1-4). In urine samples, urea is a major inhibitor which directly interacts with and degrades polymerases (5-6). Stool and fecal samples contain highly variable inhibitors dependent on lifestyle, nutrition and gut flora; resulting in polysaccharides and polyphenols originating from vegetables, urea, lipids, bile salts and hemoglobin (7-10). Among these substances, polysaccharides and polyphenols play major roles in inhibiting PCR. Complex polysaccharides co-precipitate with nucleic acids and reduce the capacity for resuspending DNA, but more importantly, polysaccharides also mimic the structure of DNA and consequently interfere with the PCR enzymatic process (11-13). On the other hand, polyphenols exert their potent inhibitory effects by directly crosslinking with nucleic acids and forming complexes that block the PCR process (11, 14-15).

There are currently two strategies for preventing or minimizing PCR inhibition. The first strategy involves the purification and isolation of DNA from source material (such as tissues and crude extracts). The isolation of DNA from sample matrices is conceptually ideal because it should separate or remove the PCR inhibitors from the DNA. In practice however, PCR inhibitors which directly bind to DNA will tend to co-purify with it, contaminating the ‘purified’ DNA sample and inhibiting downstream PCR processes. In these situations, specific PCR additives must be painstakingly screened and identified for inclusion in the PCR to overcome inhibition (2). Further, DNA purification is a multi-step process which is inefficient at multiple levels. For example, column chromatography with Sephacryl®, Sephadex®, Silica and Cation Exchange resins may be necessary for particularly troublesome samples (e.g. stool or whole blood), thereby increasing costs, processing time and loss of starting DNA material (16-20). In addition, the reagents used for efficient cell lysis and isolation of pure DNA, such as excess KCl and NaCl, EDTA, ionic detergents such as sodium deoxycholate and SDS, ethanol and isopropanol, and phenol, can also remain with the DNA sample and inhibit PCR (13, 21-24).

The second strategy circumvents the inherent disadvantages of the DNA purification process by direct PCR of tissues or crude extracts. This is achieved with robust PCR systems which use modified or chimeric DNA polymerases, and optimally modified buffer systems that enable DNA amplification in the presence of PCR inhibitors (2, 25-26). However, these systems are expensive and significantly increase costs when a large number of samples need to be analyzed. In addition, because there is a limit to the type of modifications that can be made to the DNA polymerase-buffer system, these systems are insufficiently versatile to accommodate PCR from a wide variety of sample types and PCR inhibitors. In other words, one either has to utilize different DNA-polymerase-buffer systems for different sample matrices (if they exist), or use other techniques that are far less satisfactory in overcoming PCR inhibition while waiting for a new system product that accommodates that particular type of source material.

Whole blood or plasma/serum is an attractive sample source for the direct PCR detection of DNA templates. These sample types are easily obtainable, require minimally invasive procedures with no or minimal downstream processing, and offer the possibility for the rapid and scalable detection of diseases from a large population. However, there is currently no DNA polymerase-buffer system that is able to overcome direct PCR inhibition from whole blood or plasma/serum (2, 25-26). PCR efficiency is hence limited by problems associated with the multi-step DNA isolation process, such as DNA loss, costs, time, inability to remove DNA-bound inhibitors and the introduction of other inhibitors from reagents. Consequently, in order for the direct PCR of whole blood or plasma/serum to be feasible, an alternative strategy must be utilized which focuses on materials that specifically bind to and remove PCR inhibitors inherently present in this type of sample matrix.

Four observations lie at the heart of the present disclosure. Firstly, the PCR inhibitors present in whole blood, such as hemoglobin, platelets, antibodies, serum proteins and other small molecules, exert their effects by directly binding to nucleic acids such as RNA or DNA (1-4). Secondly, purification of DNA from these inhibitors is not effective for inhibitors that bind to DNA because they co-purify with it, and remain in the DNA sample to inhibit PCR. DNA purification is also a multi-step process that is time consuming, not cost-effective and increases the loss of starting DNA material. Furthermore, the reagents used in the DNA isolation process can also remain with the DNA sample and inhibit PCR (13, 21-24). Thirdly, the development of modified DNA polymerase-buffer systems that enable direct PCR in the presence of a wide variety of PCR inhibitors is limited due to protein structural constraints and buffer compatibility. In other words, there is no DNA polymerase-buffer system that is able to overcome PCR inhibition from whole blood or plasma/serum (2, 25-26). It is also inherently expensive due to costs involved in developing the modified DNA polymerase (mutagenesis, molecular breeding or protein fusions), protein production and purification.

As such, an alternative is required that circumvents these limitations by using a “physical substrate(s)” that specifically binds to and removes or depletes PCR inhibitors and allows direct PCR to be achieved from whole blood or plasma/serum sample matrices. The present invention as disclosed herein does this.

Fourthly, for circulating DNA (e.g. PCR of ctDNA in cancer) and RNA (e.g. RT-PCR from viruses) in blood, early detection is critical for timely medical interventions towards curing or halting the progression of disease. However, these applications suffer from sensitivity issues because the RNA or DNA template is present in only minute amounts and require the purification and concentration of nucleic acids from larger volumes of whole blood or plasma/serum, respectively. The PCR inhibitors that bind to these nucleic acids will still be present to exert their inhibitory effects and thus a physical substrate(s) that specifically depletes or removes these inhibitors prior to nucleic acid concentration and purification as disclosed in the present invention herein, will thus play a major role in the successful early detection of these diseases.

### SUMMARY OF THE INVENTION

In accordance with present disclosure, the problem of removing or depleting PCR inhibitors from a complex mixture of proteins, hemoglobin, antibodies, platelets and other compounds found in whole blood or plasma/serum, is solved by using a substrate(s) that selectively binds to PCR inhibitors, and allows the rapid amplification of DNA by direct PCR in whole blood or plasma/serum samples, or the PCR and RT-PCR of purified DNA or RNA from plasma/serum or whole blood samples, respectively.

### Separation Device

The present disclosure comprises various embodiments of a PCR inhibitor separation device, all embodiments comprising: 1) a receptacle for holding whole blood, serum, or plasma samples; and, 2) a physical substrate for binding and removing the PCR inhibitors from the samples.

In a first embodiment, the separation device comprises: a polymeric physical substrate within a round bottomed micro-centrifuge tube.

In a second embodiment, the separation device comprises: a polymeric physical substrate in the form of a plurality of polymeric spheres within a round bottomed tube (e.g. a micro-centrifuge tube).

In a third embodiment, the separation device comprises: a 96-well plate with a polymeric physical substrate within each well.

In a fourth embodiment, the separation device comprises: a glass capillary rod within a round bottomed tube (e.g. a 2 mL round bottomed micro-centrifuge tube), and the polymeric physical substrate is covalently attached to the glass capillary rod.

In all embodiments of the separation device, the physical substrate(s) specifically depletes or removes PCR inhibitors prior to direct PCR of sample matrices or nucleic acid concentration and purification. In an embodiment, PBS buffer (e.g. 137 mM NaCl, 2.7 mM KCl, 10 mM Na₂HPO₄, 1.8 mM KH₂PO₄, pH7.4) is added to the separation device before or after adding the physical substrate, allowed to equilibrate with the substrate for an appropriate amount of time, and the buffer subsequently removed prior to adding the patient blood or serum sample.

### Method of Use of Separation Device

The present disclosure further comprises a method of removing inhibitor(s) of a polymerase chain reaction (PCR) from sample matrices, by: 1) contacting a sample matrix (i.e. a patient sample) with a physical substrate in such a manner as to bind and deplete said PCR inhibitor(s) from the sample matrix, wherein a plurality of nucleic acids within the sample matrix does not bind to the physical substrate; and 2) recovering the sample matrix with the plurality of nucleic acids after said depletion of the PCR inhibitor(s).

The present disclosure further comprises a method of use of the separation device comprising: 1) providing a separation device comprising: a) a receptacle for holding whole blood, serum, or plasma samples; and, b) a physical substrate located within the receptacle, for binding and removing the PCR inhibitors from the samples; 2) contacting sample matrices (i.e. a patient sample) with a physical substrate in such a manner as to bind and deplete said PCR inhibitor(s) from the sample matrices, wherein a plurality of nucleic acids within the sample matrices does not bind to the physical substrate; and 3) recovering the sample matrices with the plurality of nucleic acids after said depletion of the PCR inhibitor(s).

In one embodiment, the separation device receptacle is treated with PBS buffer, such as 137 mM NaCl, 2.7 mM KCl, 10 mM Na₂HPO₄, 1.8 mM KH₂PO₄, pH7.4, before the sample matrices (e.g. patient sample) is added to the separation device. PBS buffer is added to the separation device before or after adding the physical substrate, allowed to equilibrate with the substrate for an appropriate amount of time, and the buffer subsequently removed prior to adding the patient blood or serum sample.

In another embodiment, whole blood is added to ethylene-diamine-tetraacetic acid (EDTA) treated plastic tubes and inverted several times to prevent coagulation. The EDTA-treated blood is then stored at 4° C.

In another embodiment, whole blood is centrifuged at 10,000 rpm for 10 mins, and the plasma supernatant is transferred to fresh tubes and stored at 4° C.

### Physical Substrates

In all embodiments disclosed herein, PCR inhibitors in the patient sample (e.g. whole blood or plasma/serum) bind to the physical substrate located within the receptacle of the separation device; but the DNA/RNA within the patient sample does not bind to the physical substrate.

In an embodiment, the physical substrate comprises a macromolecular matrix; and/or is in the shape of: a dome, sphere, sheet, pieces, slurry or any similar configuration.

In an embodiment, the physical substrate comprises an acrylamide based polymer that is made by mixing acrylamide, acrylic acid, N,N′-Methylenebisacrylamide, N-isopropyl acrylamide, N(1,1-dimethyl-3-oxobutyl) acrylamide and 2-methacryloxyethyl phenyl urethane in their appropriate amounts in a container, such as a 2 mL round bottomed micro centrifuge tube or a 96 well plate, and adding 1 volume of DMPA photo-initiator, and subsequently polymerizing under UV light.

In another embodiment, the physical substrate comprises of a non-acrylamide based polymer, such as sodium alginate or polyvinyl alcohol, made by mixing the co-polymers of sodium alginate, lambda carrageenan and polyvinyl alcohol in their appropriate amounts and dissolving them in a base polymer of melted agarose, and then cooling until hardened to a gel.

In another embodiment, the physical substrate(s) is a macromolecular matrix in the form of a polymer, comprising one or more of: sodium alginate, carrageenan, chitin, starch, polyvinyl alcohol, hydroxyethyl cellulose, hydroxypropyl cellulose and agarose.

In another embodiment, the physical substrate(s) comprises agarose, and one or more of: sodium alginate, lambda carrageenan and polyvinyl alcohol.

In another embodiment, the physical substrate(s) comprises agarose, lambda carrageenan, hydroxyethyl cellulose and hydroxypropyl cellulose.

In another embodiment, the physical substrate is a crosslinked copolymer comprising: acrylamide and N,N′-Methylenebisacrylamide, and one or more derivatives of acrylamide and acrylate, and glycerol.

In another embodiment, the physical substrate is a macromolecular matrix of crosslinked copolymers comprising, 1) acrylamide and N,N′-Methylenebisacrylamide; and/or 2) one or more monomers selected from a group consisting of: acrylic acid, N-isopropyl acrylamide, N(1,1-dimethyl-3-oxobutyl) acrylamide, 2-methacryloxyethyl phenyl urethane; and glycerol.

### Kits

The present disclosure further comprises a kit comprising consumables and instructions for separating PCR inhibitor(s) from whole blood, serum and plasma samples, and recovery of the nucleic acid containing components. The kit further comprises a receptacle for holding the whole blood, serum or plasma sample and the physical substrate.

The nucleic acids containing components used with the kit comprise one or more of: cells, viral capsids, exosomes and free-floating nucleic acids found in the sample matrices, and said nucleic acids containing components are recovered by transferring the samples to new receptacles after incubation with the physical substrate.

The physical substrate within the kit is a macromolecular matrix; and/or takes the shape of a dome, sphere, sheet, pieces, slurry or any similar configuration.

The receptacle within the kit comprises, a plastic micro-centrifuge tube, a plastic 96-well plate, a glass vial, a glass test tube, a plastic test tube, or any similar receptacle; and may be pre-treated with PBS buffer.

Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

For a more complete understanding of the invention, reference is made to the following description and accompanying drawings, in which:

FIGS. 1A and 1B illustrate the plastic micro-centrifuge tube polymer separation device containing the polymer in the shape of a dome.

FIG. 1A comprises the first embodiment of the separation device comprising a round bottom micro centrifuge tube holding the polymeric physical substrate in PBS buffer (e.g. 137 mM NaCl, 2.7 mM KCl, 10 mM Na₂HPO₄, 1.8 mM KH₂PO₄, pH7.4). The PBS buffer is subsequently removed prior to addition of the blood sample.

FIG. 1B comprises the separation device with a total volume of 200 uL of whole blood-PBS (1 volume of whole blood diluted with 1 volume of PBS buffer) with genomic DNA at 2 ng/uL added to the device and incubated at room temperature for 10 mins. The samples are then removed and transferred to new tubes for storage at 4° C., or immediately used in direct PCR.

FIG. 2 is a second embodiment of the plastic micro-centrifuge tube separation device containing polymer spheres as the physical substrate and in PBS buffer (e.g. 137 mM NaCl, 2.7 mM KCl, 10 mM Na₂HPO₄, 1.8 mM KH₂PO₄, pH7.4). The PBS buffer is subsequently removed and a total volume of 2 mL of whole blood-PBS (1 volume of whole blood diluted with 1 volume of PBS buffer) with genomic DNA at 2 ng/uL is then added to the device and incubated at room temperature for 10 mins. The samples are then removed and transferred to new tubes for storage at 4° C., or immediately used in direct PCR.

FIG. 3 is an overhead-top view of a third embodiment of the separation device comprising of a 96-well plate with a polymeric physical substrate within each well of the separation device. The polymeric physical substrate is equilibrated with PBS buffer; and the PBS buffer is subsequently removed prior to addition of the sample. A total volume of 50 uL of whole blood-PBS (1 volume of whole blood diluted with 1 volume of PBS buffer) with genomic DNA at 2 ng/uL is added to the separation device and incubated at room temperature for 10 mins. The samples are then removed and transferred to new tubes for storage at 4° C., or immediately used in direct PCR.

FIG. 4A is a side elevation view of a fourth embodiment comprising a glass capillary rod as part of the polymer separation device. The polymeric physical substrate is covalently attached to a glass capillary tube and immersed in PBS buffer by placing it within the 2 mL round bottomed micro-centrifuge tube sample container. After 10 mins, the PBS buffer is then removed from the sample container prior to the addition of the whole blood sample. Alternatively, the glass capillary tube covalently attached to the polymeric physical substrate is removed, flicked by hand a couple of times to remove excess PBS buffer and transferred to another micro-centrifuge tube containing the blood sample.

FIG. 4B is a side elevation view of the fourth embodiment of the glass capillary rod in a polymer separation device. A total volume of 500 uL of whole blood-PBS (1 volume of whole blood diluted with 1 volume of PBS buffer) with genomic DNA at 2 ng/uL is added to the separation device, and incubated at room temperature for 10 mins. The samples are then removed and transferred to new tubes for storage at 4° C. or immediately used in direct PCR.

FIG. 5 shows the agarose gel analysis of whole blood-PBS (2 ng/uL DNA) cleaned up with the polymer dome separation device shown in FIG. 1. Lane 1 is the positive control PCR containing only genomic DNA (1 uL of 2 ng/uL DNA dissolved in AE buffer) and shows the GAPDH DNA band. Lanes 2-8 are whole blood supernatant cleaned up with the polymer, and added in increasing amounts to the PCR reaction (total volume is 30 uL). Lanes 2-8 are 0.1 uL, 0.3 uL, 0.5 uL, 0.7 uL, 1 uL, 3 uL and 5 uL. Lane 8 at 5 uL is the only volume that leads to a small amount of GAPDH DNA (faint GAPDH DNA band).

FIG. 6 is the negative control and shows the agarose gel of whole blood-PBS (2 ng/uL DNA) that is not incubated or no cleanup with the polymer. Lane 1 is the positive control PCR containing only genomic DNA (1 uL of 2 ng/uL DNA dissolved in AE buffer) and shows the GAPDH DNA band. Lanes 2-8 are 0.1 uL, 0.3 uL, 0.5 uL, 0.7 uL, 1 uL, 3 uL and 5 uL of whole blood added to the PCR (total volume is 30 uL), and shows unsuccessful PCR with no GAPDH DNA band.

FIG. 7 depicts the agarose gel analysis of 2 mL whole blood-PBS (2 ng/uL) cleaned up with the polymer sphere separation device shown in FIG. 2. Lane 1 is the positive control PCR containing only genomic DNA (1 uL of 2 ng/uL DNA dissolved in AE buffer) and shows the GAPDH DNA band. Lanes 2-5 are whole blood supernatant cleaned up with the polymer, and added in increasing amounts to the PCR reaction (total volume is 30 uL). Lanes 2-5 are 1 uL, 3 uL, 5 uL and 7 uL added to the PCR reaction and shows the successful amplification of the GAPDH DNA band.

FIG. 8 is the negative control and shows the agarose gel analysis of 2 mL of whole blood-PBS (2 ng/uL DNA) that is not incubated or no cleanup with the polymer. Lane 1 is the positive control PCR containing only genomic DNA (1 uL of 2 ng/uL DNA dissolved in AE buffer) and shows the GAPDH DNA band. Lanes 2-5 are 1 uL, 3 uL, 5 uL and 7 uL of whole blood added to the PCR (total volume is 30 uL), and shows unsuccessful PCR with no GAPDH DNA band.

FIGS. 9A and 9B are plasma processed in the polymer dome separation device as depicted in FIG. 1. The amount of 0.5-1 mL of whole blood-PBS (1 volume whole blood diluted with 1 volume of PBS Buffer) with genomic DNA (final concentration of 2 ng/uL) is centrifuged for 10 mins and the plasma supernatant then transferred to a new tube. 200 uL of plasma-DNA is then added to the device and incubated at room temperature for 10 mins. The samples are then removed and transferred to new tubes for storage at 4° C. or immediately used in direct PCR.

FIG. 9A is Plasma-DNA with no incubation or no cleanup with the polymer.

FIG. 9B is Plasma-DNA incubated with or cleaned up with the polymer. The plasma that is processed with the polymer in FIG. 9B shows a distinct change in color and texture from the untreated plasma in FIG. 9A that is indicative of removal of substances or PCR inhibitors from the plasma.

FIG. 10 shows the agarose gel analysis of plasma (2 ng/uL DNA) cleaned up with the polymer. Lane 1 is the positive control PCR containing only genomic DNA (1 uL of 2 ng/uL DNA dissolved in AE buffer) and shows the GAPDH DNA band. Lanes 2-7 are plasma supernatant cleaned up with the polymer, and added in increasing amounts to the PCR reaction (total volume is 30 uL). Lanes 2-7 are 0.1 uL, 0.5 uL, 1 uL, 3 uL, 5 uL and 10 uL. The results show a progressive increase in the GAPDH DNA band that corresponds to the progressive increase of input cleaned-up plasma in the PCR reaction. In other words, the plasma has been successfully depleted of PCR inhibitors by the polymer.

FIG. 11 is the negative control and shows the agarose gel of plasma (2 ng/uL DNA) that is not incubated or no cleanup with the polymer. Lane 1 is the positive control PCR containing only genomic DNA (1 uL of 2 ng/uL DNA dissolved in AE buffer) and shows the GAPDH DNA band. Lanes 2-7 are 0.1 uL, 0.5 uL, 1 uL, 3 uL, 5 uL and 10 uL of plasma added to the PCR (total volume is 30 uL), and shows unsuccessful PCR with no GAPDH DNA band.

### DETAILED DESCRIPTION OF THE INVENTION

Various embodiments of the present disclosure are described more fully hereinafter with reference to the accompanying figures, in which some, but not all embodiments are shown in the figures. Indeed, these inventions may be embodied in different forms and should not be construed as limited to the embodiments set forth herein; rather, these embodiments are provided so that this disclosure will satisfy applicable legal requirements.

### Glossary of Terms

Unless otherwise defined herein, all terms used in this application are to be afforded the usual meaning in the art, as they would be understood by a person of ordinary skill at the time of invention. It should be understood that throughout this application singular forms, such as “a”, “an”, and “the” are often used for convenience; however, singular forms are intended to include the plural unless specifically limited to the singular either explicitly or by context.

“Macromolecules” include oligomers, polymers, dendrimers, nanospheres, nanotubes and the like.

“Macromolecular matrices” that are non-acrylamide based physical substrates comprise of one or more of: sodium alginate, carrageenan, chitin, starch, polyvinyl alcohol, hydroxyethyl cellulose, hydroxypropyl cellulose and agarose.

“Macromolecular matrices” that are acrylamide based physical substrates comprise of one or more of: acrylamide, N,N′-Methylenebisacrylamide, acrylic acid, N-isopropyl acrylamide, N(1,1-dimethyl-3-oxobutyl) acrylamide, 2-methacryloxyethyl phenyl urethane and glycerol.

“Small molecules” and “substances” include biologically or environmentally relevant molecules having a molecular weight lower than that of macromolecules.

“Nucleic acids” are ribonucleic acid (RNA) and deoxyribonucleic acid (DNA) molecules.

“Whole Blood” is blood collected from donors and added to EDTA-treated blood collection tubes (such as the BD 367856 Vacutainer Plus EDTA Blood Collection Tubes), and immediately inverted several times to prevent coagulation. The EDTA-treated whole blood is then stored at 4° C. without any further processing until added to the separation device disclosed herein.

Whole Blood-PBS is 1 volume of whole blood diluted with 1 volume of PBS buffer and is the sample matrix used for incubation with the polymers.

“Plasma” is obtained from whole blood (no PBS dilution) that has been centrifuged to separate the insoluble red blood and white blood cells from the soluble plasma. Upon completion of centrifugation, the plasma supernatant is then transferred to fresh tubes for immediate incubation with the polymers or stored at 4° C.

“Silane solution” is a silanizing agent consisting of 3-methacryloxy propyl trimethylsilane or 3-acryloxy propyl trimethylsilane mixed with absolute ethanol and glacial acetic acid. It is used to silanize glass surfaces (such as glass capillary tubes) for subsequent covalent attachment of polymers under UV exposure.

“DMPA” is 2,2-dimethoxy-2-phenylacetophenone and is a photo-initiator that produces free radicals upon UV light exposure resulting in polymerization of acrylamide-based polymers.

“DWG solvent” consists of DMSO, water and glycerol in a ratio of 88:10:2 and is the solvent used to dissolve the DMPA photo-initiator powder.

“DMPA Photo-Initiator Solution” is 20 to 50 mg of DMPA dissolved in DWG solvent. One volume of this solution is then added to the acrylamide based monomer mix to enable polymerization under UV light exposure.

“PBS’ is a Phosphate Saline Buffer (137 mM NaCl, 2.7 mM KCl, 10 mM Na₂HPO₄, 1.8 mM KH₂PO₄, pH7.4) used for equilibrating the substrate or polymer and diluting the whole blood samples.

“AE” is a Tris EDTA buffer (10 mM Tris-HCl, 0.5 mM EDTA, pH 9) and is used for preparing and storing the genomic DNA at −80° C. This genomic DNA is the DNA template used for the positive control PCR which leads to the GAPDH band observed in agarose gels.

“TAE” is a Tris Acetate buffer commonly used in agarose gel electrophoresis (40 mM Tris-HCl, 20 mM Acetic Acid, 1 mM EDTA, pH 8.3).

“PCR” is the polymerase chain reaction in which DNA is amplified with thermostable DNA polymerases using specific primers

“5×PCR Mix” contains the Buffer, dNTPs and Taq polymerase at 5X concentration. “RT-PCR” is a variant of the PCR process in which RNA is first transcribed into DNA by reverse transcriptase, and the resulting DNA then amplified with thermostable DNA polymerases using specific primers.

“10×RT Buffer” is the standard or optimal buffer used for reverse transcription reactions and consists of 500 mM Tris-HCl pH8.3, 750 mM KCl, 30 mM MgCl₂, 100 mM DTT.

“RNase Inhibitor” is an additive that inhibits RNase and prevents the degradation of the total RNA or mRNA template.

“Random nonamer N9 primers” are small DNA containing random sequences that randomly primes different regions of the total RNA template, resulting in a range of first strand DNA sizes corresponding to different regions (and genes) of the original genomic DNA template.

“Poly dT primers” are small DNA containing dT sequences that specifically binds to the poly A tail of messenger RNA (mRNA) and enables first strand DNA synthesis of the mRNA template.

“dNTPs” are equal concentrations of the 4 deoxynucleotides (dATP, dGTP, dTTP, dCTP) that are the building blocks for first strand DNA synthesis.

“GAPDH For and GAPDH Rev primers” are small DNA containing sequences specific to the GAPDH gene and allows PCR DNA amplification of the GAPDH gene. The GAPDH for primer consists of the sequence 5′ACCACAGTCCATGCCATCAC3′ and the GAPDH Rev primer consists of the sequence 5′ TCCACCACCCTGTTGCTGTA3′.

“Placental DNA” is human placental genomic DNA that is used as a template for the GAPDH primers and PCR amplification of the GAPDH gene.

For acrylamide based polymers, the physical substrate 110 or 410 is acquired by mixing the monomers of N-isopropyl acrylamide, N(1,1-dimethyl-3-oxobutyl) acrylamide, acrylic acid and 2-methacryloxyethyl phenyl urethane in a compositional ratio by mass of 5.2:2.3:1.9:0.6 into a container such as a 2 mL round bottomed micro centrifuge tube 100 or a 96-well plate 300, and adding 1 volume of DMPA photo-initiator solution, and subsequently polymerized under UV light.

For covalent attachment, the glass capillary tubes are treated with silane solution and dried at 110-130° C. for 1 hour. The silane-treated glass capillary tubes are then immersed in a container, such as a micro centrifuge tube that contains the appropriate polymers with DMPA solution, and simultaneous covalent attachment and polymerization are then achieved under UV light exposure.

For non-acrylamide based polymers, the physical substrate 110 or 410 is acquired by mixing the polymers of sodium alginate, lambda carrageenan and polyvinyl alcohol in a compositional ratio by mass of 8:1:1 into a container such as a glass bottle and dissolving them in a base polymer of melted agarose. The physical substrate is then obtained by cooling down and hardening at room temperature. The physical substrate gel is then re-melted and an appropriate amount (e.g. 200 uL) is then added to a 2 mL round bottomed micro centrifuge tube and allowed to re-harden for subsequent studies with whole blood or plasma samples.

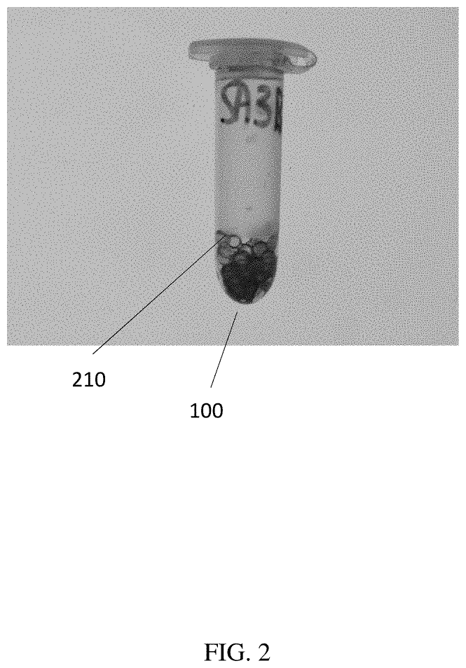

For non-acrylamide based polymer spheres (FIG. 2, 210), the physical substrate 210 is acquired by mixing the polymers of sodium alginate, lambda carrageenan and polyvinyl alcohol in a compositional ratio by mass of 8:1:1 into a container such as a glass bottle and dissolving them in a base polymer of melted agarose. The physical substrate is then obtained by cooling down and hardening at room temperature. The physical substrate gel is then re-melted and an appropriate amount (e.g. 200 uL) is then spotted onto parafilm in 5 uL aliquots and allowed to re-harden at room temperature. The gel spheres are then transferred into a 2 mL round bottomed micro centrifuge tube that contains PBS buffer for immediate usage with samples or long-term storage at 4° C.

Blood samples were collected from consented donors under an IRB-approved protocol into BD 367856 Vacutainer EDTA blood collection tubes. The tubes were inverted several times to thoroughly mix the blood with the anticoagulant EDTA, and subsequently stored at 4° C.

For the whole blood study, an aliquot of whole blood stored in BD 367856 Vacutainer EDTA blood collection tubes are transferred to a new tube and subsequently diluted with one volume of 1×PBS buffer. Placenta DNA is then added to the diluted whole blood to reach a final concentration of 2 ng/uL (Table 3), and the final blood solution is mixed thoroughly with a micro pipet and is now ready for incubation with the polymers.

TABLE 3
Preparation of whole blood samples with Genomic DNA at
a final concentration of 2 ng/uL that is incubated with or
without the polymers, and subsequently used in direct PCR.

| | 200 uL Sample | 500 uL Sample | 2000 uL Sample |
|---|---|---|---|
| | (0.2 mL) | (0.5 mL) | (2 mL) |
| Human | 4 | 10 | 40 |
| Genomic DNA | | | |
| 0.1 ug/uL | | | |
| Whole Blood- | 98 | 245 | 980 |
| EDTA uL | | | |
| 1× PBS | 98 | 245 | 980 |

For the plasma study, genomic DNA is added to whole blood to a final concentration of 2 ng/uL (Table 4). The whole blood-DNA is then centrifuged for 10 mins with the soluble plasma-DNA supernatant then transferred to a new tube and subsequently used for incubation with the polymers.

Table 4 illustrates the preparation of Plasma-DNA samples from whole blood, in which 0.5-1 mL of whole blood-DNA (2 ng/uL) is prepared according to the Table 4 protocol below and mixed thoroughly by gently pipetting up and down a few times. The Whole Blood-DNA is then centrifuged for 10 mins, and the plasma-DNA supernatant is then transferred to a new tube. The plasma-DNA is then incubated with or without the polymer for 10 mins at room temperature, and subsequently used in direct PCR.

TABLE 4
the preparation of Plasma-DNA samples from whole blood

| | 500 uL Sample | 1000 uL Sample |
|---|---|---|
| Human Genomic DNA | 10 | 20 |
| 0.1 ug/uL | | |
| Whole Blood-EDTA uL | 490 | 980 |

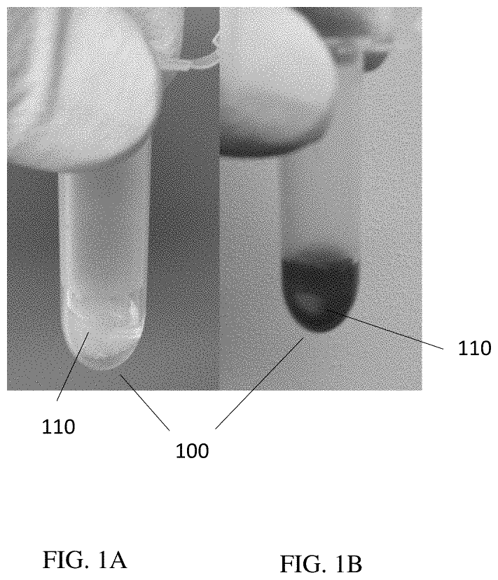

FIGS. 1A, 1B, 2, 4 are side elevational views of exemplary embodiments of the verification separation device.

In one embodiment, the vessel of the separation device 100 is a round-bottomed plastic micro-centrifuge tube containing the physical substrate polymer in PBS buffer in the shape of a gel dome and equilibrated with the buffer for 10 mins at room temperature (FIG. 1A). For the whole blood study, the PBS buffer is removed and 200 uL of diluted whole blood-PBS-DNA (2 ng/uL) is added to the polymer and incubated at room temperature for 10-15 mins (FIG. 1B). The blood samples are then removed and transferred to new tubes, and subsequently used at 0.1-5 uL for PCR (Table 5). Control samples consist of diluted whole blood-PBS-DNA (2 ng/uL), but these controls are not incubated with the polymer and are instead used directly in the PCR at 0.1-5 uL (Table 6).

TABLE 5
PCR of whole blood samples incubated or cleaned up with polymers. A total volume
of 30 uL was used in the PCR analysis.

| Amount | Sample | Sample | Sample | Sample | Sample | Sample | Sample | Sample |
|---|---|---|---|---|---|---|---|---|
| used in | Cleanup0. | Cleanup0. | Cleanup0. | Cleanup0. | Cleanup | Cleanup | Cleanup | Cleanup |
| PCR | 1 | 3 | 5 | 7 | 1 | 3 | 5 | 7 |
| Whole | 1 uL | 3 uL | 5 uL | 7 uL | | | | |
| Blood | | | | | | | | |
| Diluted | | | | | | | | |
| 1/10 with | | | | | | | | |
| AE Buffer | | | | | | | | |
| Whole | | | | | 1 uL | 3 uL | 5 uL | 7 uL |
| Blood | | | | | | | | |
| GAPDH | 1 uL | 1 uL | 1 uL | 1 uL | 1 uL | 1 uL | 1 uL | 1 uL |
| For | | | | | | | | |
| Primer | | | | | | | | |
| (5 pmol/uL) | | | | | | | | |
| GAPDH | 1 uL | 1 uL | 1 uL | 1 uL | 1 uL | 1 uL | 1 uL | 1 uL |
| Rev | | | | | | | | |
| Primer | | | | | | | | |
| (5 pmol/uL) | | | | | | | | |
| 5X PCR | 6 uL | 6 uL | 6 uL | 6 uL | 6 uL | 6 uL | 6 uL | 6 uL |
| Mix | | | | | | | | |
| Water | 21 uL | 19 uL | 17 uL | 15 uL | 21 uL | 19 uL | 17 uL | 15 uL |

TABLE 6
PCR of whole blood samples not incubated or without clean-up with the polymers.
A total volume of 30 uL was used in the PCR analysis.

| | | Negative | Negative | Negative | Negative | Negative | Negative | Negative | Negative |
|---|---|---|---|---|---|---|---|---|---|
| Amount in | Positive | Control0 | Control0. | Control0. | Control0. | Control | Control | Control | Control |
| PCR | Control | .1 | 3 | 5 | 7 | 1 | 3 | 5 | 7 |
| DNA | 1 uL | | | | | | | | |
| (2 ng/uL) in | | | | | | | | | |
| AE Buffer | | | | | | | | | |
| Whole | | 1 uL | 3 uL | 5 uL | 7 uL | | | | |
| Blood | | | | | | | | | |
| Diluted | | | | | | | | | |
| 1/10 with | | | | | | | | | |
| AE Buffer | | | | | | | | | |
| Whole | | | | | | 1 uL | 3 uL | 5 uL | 7 uL |
| Blood | | | | | | | | | |
| GAPDH | 1 uL | 1 uL | 1 uL | 1 uL | 1 uL | 1 uL | 1 uL | 1 uL | 1 uL |
| For | | | | | | | | | |
| Primer | | | | | | | | | |
| 5 pmol/uL | | | | | | | | | |
| GAPDH | 1 uL | 1 uL | 1 uL | 1 uL | 1 uL | 1 uL | 1 uL | 1 uL | 1 uL |
| Rev | | | | | | | | | |
| Primer | | | | | | | | | |
| 5 pmol/uL | | | | | | | | | |
| 5X PCR | 6 uL | 6 uL | 6 uL | 6 uL | 6 uL | 6 uL | 6 uL | 6 uL | 6 uL |
| Mix | | | | | | | | | |
| Water | 21 uL | 21 uL | 19 uL | 17 uL | 15 uL | 21 uL | 19 uL | 17 uL | 15 uL |

In another embodiment, the vessel of the separation device is a round-bottomed plastic micro-centrifuge tube 100 and containing the physical substrate in the shape of polymeric gel spheres (FIG. 2, 210). The polymeric gel spheres were equilibrated with PBS buffer for 10 mins at room temperature. For the whole blood study, the PBS buffer is removed and 2000 uL or 2 mL of whole blood-PBS-DNA (2 ng/uL) is added to the polymer and incubated at room temperature for 10-15 mins. The blood samples are then removed and transferred to new tubes, and subsequently used at 1-7 uL for PCR (Table 5). Control samples consist of diluted whole blood-PBS-DNA (2 ng/uL), but these controls are not incubated with the polymer and are instead used directly in the PCR at 1-7 uL (Table 6).

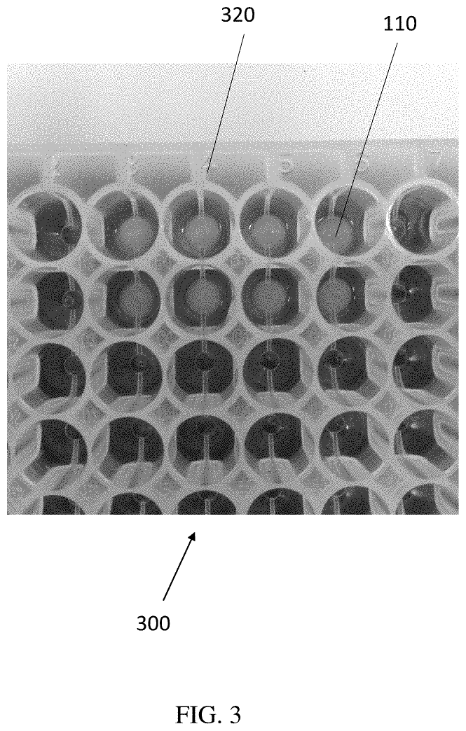

In another embodiment, the separation device 300 consists of a 96-well plate containing the physical substrate polymer 110 within the wells. The polymer is equilibrated with PBS buffer for 10 mins at room temperature and subsequently removed prior to the addition of whole blood samples (FIG. 3, 320 wells). For the whole blood study, 50 uL of diluted whole blood-PBS-DNA (2 ng/uL) is added to the polymer and incubated at room temperature for 10-15 mins. The blood samples are then removed and transferred to new tubes, and subsequently used at 0.1-5 uL for PCR (Table 5). Control samples consist of diluted whole blood-PBS-DNA (2 ng/uL), but these controls are not incubated with the polymer and are instead used directly in the PCR at 0.1-5 uL (Table 6).

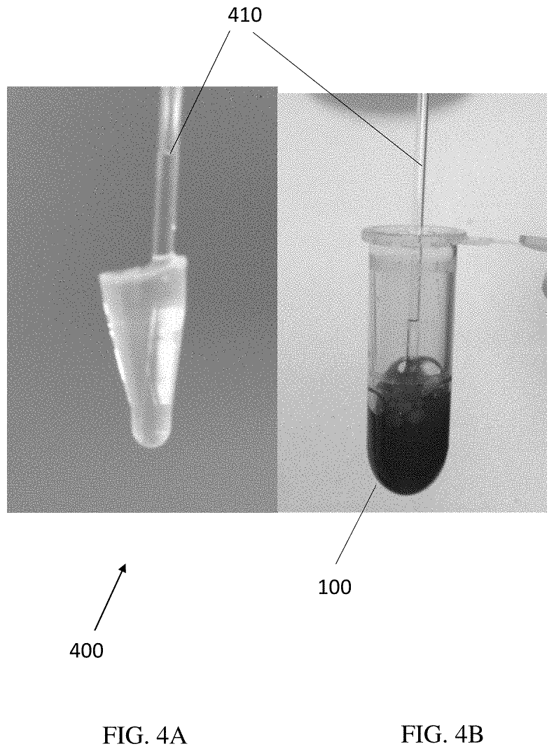

In another embodiment, the separation device 400 consists of the polymer covalently attached to a glass capillary tube 410, and the container of the device is a 2 mL round bottomed micro centrifuge tube 100 (FIG. 4). The covalently attached polymer is equilibrated with PBS buffer for 10 mins at room temperature in the 2 mL round bottomed micro centrifuge tube. For the whole blood study, the capillary tube polymer is taken out of the micro centrifuge tube and flicked by hand a few times to remove excess PBS buffer and transferred to another 2 mL round bottomed micro centrifuge tube containing 500 uL of diluted whole blood-PBS-DNA (2 ng/uL), and incubated at room temperature for 10-15 mins. The blood samples are then removed and transferred to new tubes, and subsequently used at 0.1-5 uL for PCR (Table 5). Control samples consist of diluted whole blood-PBS-DNA (2 ng/uL), but these controls are not incubated with the polymer and are instead used directly in the PCR at 0.1-5 uL (Table 6).

In one embodiment of the plasma study, the vessel of the separation device is a round-bottomed plastic micro centrifuge tube 100 containing the physical substrate polymer in PBS buffer in the shape of a gel dome and equilibrated with the buffer for 10 mins at room temperature (FIGS. 1A, 1B). For the plasma study, the PBS buffer is subsequently removed and 100 uL of plasma-DNA (2 ng/uL) is added to the polymer and incubated at room temperature for 10 mins. The plasma samples are then removed and transferred to new tubes, and subsequently used at 0.1-10 uL for PCR (Table 7). Control samples consist of plasma-DNA (2 ng/uL), but these controls are not incubated with the polymer and are instead used directly in the PCR at 0.1-10 uL (Table 8).

TABLE 7
PCR of Plasma samples incubated or cleaned up with polymers. A total volume of 30 uL was
used in the PCR analysis.

| Amount used | Sample | Sample | Sample | Sample | Sample | Sample |
|---|---|---|---|---|---|---|
| in PCR | Cleanup 0.1 | Cleanup 0.5 | Cleanup 1 | Cleanup 3 | Cleanup 5 | Cleanup 10 |
| Plasma | 1 uL | 5 uL | | | | |
| Diluted 1/10 | | | | | | |
| with AE | | | | | | |
| Buffer | | | | | | |
| Plasma | | | 1 uL | 3 uL | 5 uL | 10 uL |
| GAPDH For | 1 uL | 1 uL | 1 uL | 1 uL | 1 uL | 1 uL |
| Primer | | | | | | |
| (5 pmol/uL) | | | | | | |
| GAPDH Rev | 1 uL | 1 uL | 1 uL | 1 uL | 1 uL | 1 uL |
| Primer | | | | | | |
| (5 pmol/uL) | | | | | | |
| 5X PCR Mix | 6 uL | 6 uL | 6 uL | 6 uL | 6 uL | 6 uL |
| Water | 21 uL | 17 uL | 21 uL | 19 uL | 17 uL | 12 uL |

TABLE 8
PCR of plasma samples not incubated or without clean-up with the polymers. A total
volume of 30 uL was used in the PCR analysis.

| Amount in | Positive | Negative | Negative | Negative | Negative | Negative | Negative |
|---|---|---|---|---|---|---|---|
| PCR | Control | Control 0.1 | Control 0.5 | Control 1 | Control 3 | Control 5 | Control10 |
| DNA | 1 uL | | | | | | |
| (2 ng/uL) in | | | | | | | |
| AE Buffer | | | | | | | |
| Plasma | | 1 uL | 5 uL | | | | |
| Diluted 1/10 | | | | | | | |
| with AE | | | | | | | |
| Buffer | | | | | | | |
| Plasma | | | | 1 uL | 3 uL | 5 uL | 10 uL |
| GAPDH | 1 uL | 1 uL | 1 uL | 1 uL | 1 uL | 1 uL | 1 uL |
| For Primer | | | | | | | |
| (5 pmol/uL) | | | | | | | |
| GAPDH | 1 uL | 1 uL | 1 uL | 1 uL | 1 uL | 1 uL | 1 uL |
| Rev Primer | | | | | | | |
| (5 pmol/uL) | | | | | | | |
| 5X PCR | 6 uL | 6 uL | 6 uL | 6 uL | 6 uL | 6 uL | 6 uL |
| Mix | | | | | | | |
| Water | 21 uL | 21 uL | 17 uL | 21 uL | 19 uL | 17 uL | 12 uL |

Reverse Transcription (RT) is conducted according to established procedures. A standard RT protocol consists of (total volume of 20 uL):

| | | |
|---|---|---|
| Total RNA or Poly A-selected mRNA | 0.5-2 | ug |
| Random nonamer (N9) or Poly dT primer 40 uM | 2 | uL |
| 2.5 mM dNTP | 4 | uL |
| RNase-free water to a total volume of 16 uL | | |

Heat at 65° C.-80° C. for 3-5mins and immediately
place the tube(s) on ice.

| | | |
|---|---|---|
| Then add: | | |
| 10× RT Buffer | 2 | uL |
| RNase Inhibitor (10 U/uL) | 1 | uL |
| M-MuLV Reverse Transcriptase (200 U/uL) | 1 | uL |
| Incubate at 42° C. for 1 hour. | | |
| Inactivate enzyme at 90° C. for 10 mins. | | |
| Products are stored at −20° C. or used directly for PCR. | | |

PCR of processed samples is conducted according to established PCR protocols. The direct PCR protocol used in this study is shown in Tables 5 to 8 (total volume of 30 uL).

The Direct PCR parameters used in this study consists of:

| | |
|---|---|
| 95° C. | 180 s Initial DNA Denaturation |
| 95° C. | 30 s |
| 55° C. | 30 s |
| 72° C. | 30 s |
| 30 cycles | |
| 72° C. | 60 s Final Extension |
| 4° C. | Hold |

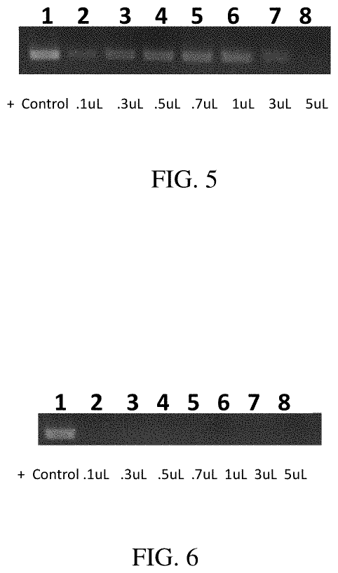

Agarose gel electrophoresis is conducted according to established protocols. 2 uL of loading buffer was added to the PCR samples, and 10 uL of the whole blood or plasma and control PCR samples are then loaded into the wells of the agarose gel. The gel was then run at 120 V in 1×TAE buffer. Upon completion, the DNA band consisting of the GAPDH gene is visualized with UV illumination, and a photograph is taken with a digital camera (FIGS. 5-8 and 10-11).

For the smaller volume or 200 uL whole blood study shown in FIG. 5, Lane 1 is the positive control PCR with 1 uL of placenta DNA (2 ng/uL) only and shows the GAPDH DNA band (part of the GAPDH gene) amplified with the GAPDH primers. Lanes 2-8 are whole blood samples cleaned up with the polymer and added to the PCR in increasing amounts of 0.1 uL, 0.3 uL, 0.5 uL, 0.7 uL, 1 uL, 3 uL and 5 uL (Table 5). The result shows that the polymer effectively removes whole blood PCR inhibitors resulting in the GAPDH DNA band (only Lane 8 or 5 uL has a faint band or low amounts of GAPDH DNA). For the negative control whole blood study in FIG. 6, Lane 1 is the positive control PCR with 1 uL of placenta DNA (2 ng/uL) only and shows the GAPDH DNA band (part of the GAPDH gene) amplified with the GAPDH primers. Lanes 2-8 are whole blood samples without clean up or not incubated with the polymer and added to the PCR in increasing amounts of 0.1 uL, 0.3 uL, 0.5 uL, 0.7 uL, 1 uL, 3 uL and 5 uL (Table 6). The results show the presence and potency of the whole blood PCR inhibitors leading to unsuccessful PCR with no GAPDH bands for all samples.

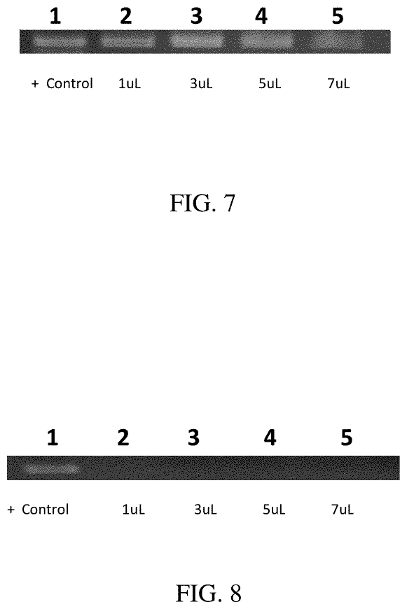

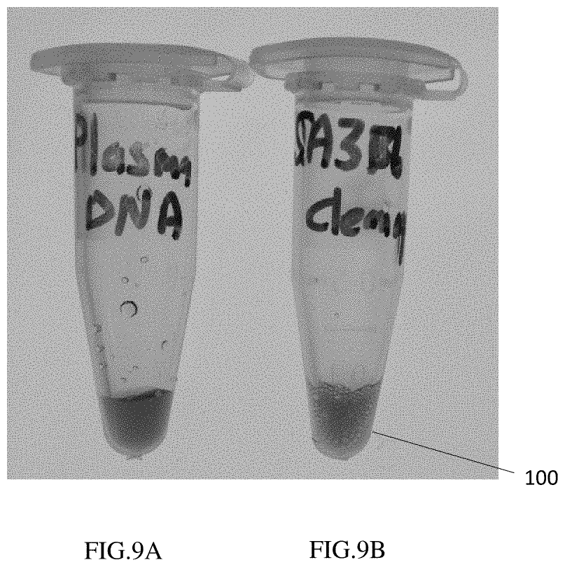

For the larger volume or 2 mL whole blood study shown in FIG. 7, Lane 1 is the positive control PCR with 1 uL of placenta DNA (2 ng/uL) only and shows the GAPDH DNA band (part of the GAPDH gene) amplified with the GAPDH primers. Lanes 2-5 are whole blood samples cleaned up with the polymer and added to the PCR in increasing amounts of 1 uL, 3 uL, 5 uL and 7 uL (Table 5). The result shows that the polymer effectively removes or depletes whole blood PCR inhibitors resulting in the GAPDH DNA band for all samples. For the negative control 2 mL whole blood study in FIG. 8, Lane 1 is the positive control PCR with 1 uL of placenta DNA (2 ng/uL) only and shows the GAPDH DNA band (part of the GAPDH gene) amplified with the GAPDH primers. Lanes 2-5 are whole blood samples without clean up or not incubated with the polymer and added to the PCR in increasing amounts of 1 uL, 3 uL, 5 uL and 7 uL (Table 6). The results again confirm the presence and potency of the whole blood PCR inhibitors leading to unsuccessful PCR or no GAPDH DNA bands for all samples.

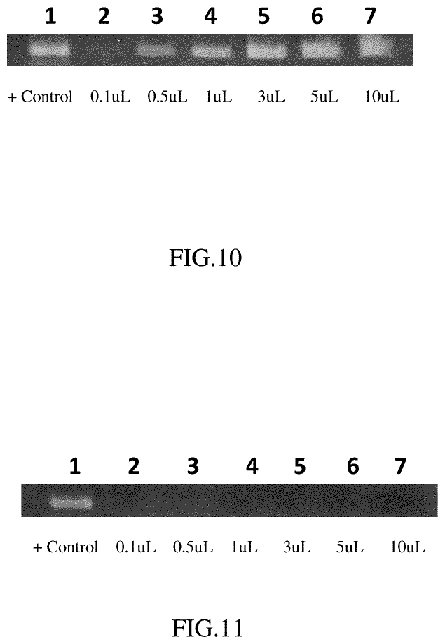

For the plasma study in FIG. 10, Lane 1 is the positive control PCR with 1 uL of placenta DNA (2 ng/uL) only and shows the GAPDH DNA band (part of the GAPDH gene) amplified with the GAPDH primers. Lanes 2-7 are plasma samples cleaned up with the polymer and added to the PCR in increasing amounts of 0.1 uL, 0.5 uL, 1 uL, 3 uL, 5 uL and 10 uL (Table 7). The result shows that the polymer effectively depletes plasma PCR inhibitors resulting in the progressive increase of the GAPDH band that corresponds to the progressive increase in input plasma sample in the PCR reaction.

For the negative control plasma study in FIG. 11, Lane 1 is the positive control PCR with 1 uL of placenta DNA (2 ng/uL) only and shows the GAPDH DNA band (part of the GAPDH gene) amplified with the GAPDH primers. Lanes 2-7 are plasma samples without clean up or not incubated with the polymer and added to the PCR in increasing amounts of 0.1 uL, 0.5 uL, 1 uL, 3 uL, 5 uL and 10 uL (Table 8). The results show that the PCR inhibitors that are present in plasma are extremely potent resulting in unsuccessful PCR with no GAPDH bands for all samples.

### REFERENCES

1. Al-Soud, W. A. et al. (2000). J Clin. Microbiol 38, 4463-4470.
2. Al-Soud, W. A. and Radstrom, P. (2001) J Clin Microbiol 39, 485-493.
3. Costafreda, M. I. et al. (2006) Appl Environ Microbiology 6, 3846-3855.
4. Konet, D. S. et al. (2000) J Virol Methods 84, 95-98.
5. Saulnier, P. and Andremont, A. (1992) J Clin Microbiol 30, 2080-2083.
6. Wilson, I. G. (1997) Appl Environ Microbiology 63, 3741-3751.
7. Oikarinen, S. et al. (2009) J Clin Virology 44, 211-214.
8. Lantz, P.-G. et al. (1997) J Microbiol Methods 28, 159-167.
9. Monteiro, L. et al. (1997) J Clin Microbiol 35, 995-998.
10. Pontiroli, A. et al. (2011) PLos ONE 6, e17916.
11. Wilkins, T. A. and Smart, L. B. (1996) In a Laboratory Guide to RNA: Isolation, Analysis and Synthesis ed. Krieg, P. A. pp. 21-42. New York: Wiley-Liss, Inc.
12. Sipahioglu, H. M. et al. (1996) J Virol Methods 137, 120-124.
13. Peist, R. et al. (2001) QIAGEN News 3, 7-9.
14. John, M. E. (1992) Nucleic Acids Res. 20, 2381.
15. Opel, K. L. et al. (2010) J Forensic Sci 55, 25-33.
16. Schmidt, B. L. et al. (1996) Diagn Microbiol Infect Dis 21, 121-128.
17. Croci, L. et al. (2008) Food Anal Methods 1, 73-84.
18. Kemp, B. M. et al. (2006) J Archaeol Sci 33, 1680-1689.
19. Jacobsen, C. S. and Rasmussen, O. F. (1992) Appl Environ Microbiol 58, 2458-2462.
20. Henson, J. M. and French, R. (1993) Annu Rev Phytopathol 31, 81-109.
21. Weyant, R. S. et al. (1990) Biotechniques 9, 308-309.
22. Katcher, H. L. and Schwartz, I. (1994) Biotechniques 16, 84-92.
23. Burkardt, H. J. (2000) Clin Chem Lab Med 38, 87-91.
24. Demeke, T. and Jenkins, G. R. (2010) Anal Bio anal Chem 396, 1977-1990.
25. Kermekchiev, M. B. et al. (2009) Nucleic Acids Res 37, e40.
26. Baar, C. et al. (2011) Nucleic Acids Res 39, e51.
27. Schrader, C. et al. (2012) J Appl Microbiol 113, 1014-1026.

What is claimed is:

1. A method of removing inhibitor(s) of a polymerase chain reaction (PCR) from sample matrices, comprising,
a. providing a separation device comprising: i) a receptacle and able to hold the sample matrices; and, ii) a physical substrate treated with PBS buffer within the receptacle, and able to bind and remove the PCR inhibitor(s) from the sample matrices;
b. contacting the sample matrices with the physical substrate within the receptacle in such a manner as to bind and deplete said PCR inhibitor(s) from the sample matrices, wherein a plurality of nucleic acids containing components within the sample matrices does not bind to the physical substrate;
c. recovering the sample matrices with the plurality of nucleic acids containing components after said depletion of the PCR inhibitor(s);
d. wherein the sample matrices are a patient sample comprising one of: whole blood, plasma or serum; and
e. wherein the receptacle comprises, a plastic micro-centrifuge tube, a plastic 96-well plate, a glass vial, a glass test tube, a plastic test tube, or any similar receptacle.

2. The method of claim 1, wherein contacting the sample matrices with physical substrate further comprises incubation at room temperature for about 10 minutes.

3. The method of claim 1 or 2, wherein the nucleic acids containing components comprise, and the method further comprises amplifying the nucleic acids via PCR.

4. The method as in one of claims 1-3, wherein the physical substrate takes the shape of a dome, sphere, sheet, pieces, slurry or any similar configuration.

5. The method as in one of claims 1-4, wherein the physical substrate is a macromolecular matrix.

6. The method of claim 5, wherein the macromolecular matrix comprises one or more of: sodium alginate, carrageenan, chitin, starch, polyvinyl alcohol, hydroxyethyl cellulose, hydroxypropyl cellulose and agarose.

7. The method of claim 5, wherein the molecular matrix comprises agarose, and one or more of: sodium alginate, lambda carrageenan and polyvinyl alcohol.

8. The method of claim 7, wherein the molecular matrix comprises agarose and further consists of sodium alginate, lambda carrageenan and polyvinyl alcohol in a compositional ratio by mass ranging from 1:1:1 to 8:1:1.

9. The method of claim 8, wherein the molecular matrix comprises of a base polymer of 0.8% agarose, and further consists of a total of 1% other polymers comprising sodium alginate, lambda carrageenan and polyvinyl alcohol in a compositional ratio by mass ranging from 1:1:1 to 8:1:1.

10. The method of claim 5, wherein the molecular matrix comprises: agarose, lambda carrageenan, hydroxyethyl cellulose and hydroxypropyl cellulose.

11. The method of claim 10, wherein the molecular matrix comprises agarose and further consists of lambda carrageenan, hydroxyethyl cellulose and hydroxypropyl cellulose in a compositional ratio by mass ranging from 1:1:1 to 2:1:1 or 1:1:1 to 1:2:1 or 1:1:1 to 1:1:2.

12. The method of claim 11, wherein the molecular matrix comprises of a base polymer of 0.8% agarose, and further consists of a total of 1% other polymers comprising lambda carrageenan, hydroxyethyl cellulose and hydroxypropyl cellulose in a compositional ratio by mass ranging from 1:1:1 to 2:1:1 or 1:1:1 to 1:2:1 or 1:1:1 to 1:1:2.

13. The method as in one of claims 1-5, wherein the physical substrate is a crosslinked copolymer comprising: glycerol, and one or more derivatives of acrylamide and acrylate.

14. The method as in one of claims 1-5, wherein the physical substrate is a crosslinked copolymer comprising: acrylamide and N,N′-Methylenebisacrylamide, and one or more derivatives of acrylamide and acrylate and glycerol.

15. The method as in one of claims 1-5 and 13, wherein the macromolecular matrix is a crosslinked copolymer comprising acrylamide and N,N′-Methylenebisacrylamide, and one or more monomers selected from a group consisting of acrylic acid, N-isopropyl acrylamide, N(1,1-dimethyl-3-oxobutyl) acrylamide, 2-methacryloxyethyl phenyl urethane and glycerol.

16. The method of claim 13, wherein the crosslinked copolymer comprises N-isopropyl acrylamide, acrylic acid and 2-methacryloxyethyl phenyl urethane, in a compositional ratio by mass ranging from 12:17:5 to 253:17:5.

17. The method of claim 13, wherein the crosslinked copolymer comprises N(1,1-dimethyl-3-oxobutyl) acrylamide, acrylic acid and 2-methacryloxyethyl phenyl urethane, in a compositional ratio by mass ranging from 6:3:1 to 22:3:1.

18. The method of claim 13, wherein the crosslinked copolymer comprises N-isopropyl acrylamide, N(1,1-dimethyl-3-oxobutyl) acrylamide, acrylic acid and 2-methacryloxyethyl phenyl urethane, in a compositional ratio by mass ranging from 6:40:10:3 to 32:6:10:3.

19. The method as in one of claims 1-5 and 13, wherein the crosslinker is N,N′-Methylenebisacrylamide.

20. A separation device comprising,
a. a receptacle able to hold sample matrices comprising whole blood, serum or plasma samples, with nucleic acids containing components; and
b. a physical substrate able to bind and remove on contact one or more PCR inhibitor(s) from the sample matrices in order to replicate the nucleic acids.

21. The separation device of claim 20, wherein the nucleic acids containing components comprise one or more of: cells, viral capsids, exosomes and free-floating nucleic acids found in the sample matrices; and said nucleic acids containing components are recovered by transferring the samples to new receptacles after incubation with the physical substrate.

22. The separation device of claim 20 or 21, wherein the physical substrate is a macromolecular matrix.

23. The separation device as in one of claims 20-22, wherein the physical substrate takes the shape of a dome, sphere, sheet, pieces, slurry or any similar configuration.

24. The separation device as in one of claims 20-23, wherein the receptacle comprises, a plastic micro-centrifuge tube, a plastic 96 well plate, a glass vial, a glass test tube, a plastic test tube, or any similar receptacle.

25. The separation device as in one of claims 20-24 is a plastic micro-centrifuge tube, and the separation device further comprises a glass capillary rod or tube with the physical substrate covalently bonded to said rod or tube.

26. A kit comprising consumables and instructions for separating PCR inhibitor(s) from whole blood, serum and plasma samples, and recovery of the nucleic acid containing components, and the kit further comprising a receptacle for holding the whole blood, serum or plasma sample and the physical substrate.

27. The kit of claim 26, wherein the nucleic acids containing components comprise one or more of: cells, viral capsids, exosomes and free-floating nucleic acids found in the sample matrices, and said nucleic acids containing components are recovered by transferring the samples to new receptacles after incubation with the physical substrate.

28. The kit of claim 26 or 27, wherein the physical substrate is a macromolecular matrix.

29. The kit as in one of claims 26-28, wherein the physical substrate takes the shape of a dome, sphere, sheet, pieces, slurry or any similar configuration.

30. The kit as in one of claims 26-29, wherein the receptacle comprises, a plastic micro-centrifuge tube, a plastic 96-well plate, a glass vial, a glass test tube, a plastic test tube, or any similar receptacle.


SEQUENCE LISTING (2 sequences)

SEQ ID NO: 1 - DNA, 20 bp, Artificial Sequence
  GAPDH forward primer
accacagtccatgccatcac

SEQ ID NO: 2 - DNA, 20 bp, Artificial Sequence
  GAPDH reverse primer
tccaccaccctgttgctgta